(19) United States
(12) Patent Application Publication
(10) Pub. No.: US 2017/0071528 A1
(43) Pub. Date: Mar. 16, 2017

(54) ADAPTABILITY SKINCARE PROMPTING SYSTEM AND METHOD

(71) Applicant: Wei-Yu CHEN, New Taipei City (TW)

(72) Inventor: Wei-Yu CHEN, New Taipei City (TW)

(21) Appl. No.: 15/308,891

(22) PCT Filed: May 19, 2014
(86) PCT No.: PCT/CN2014/000501

§ 371 Date: Nov. 4, 2016

(30) Foreign Application Priority Data
May 13, 2014 (CN) 201410197920.7

(51) Int. Cl. A61B 5/00; A45D 44/00

(52) U.S. Cl. CPC A61B 5/445; A61B 5/0075; A61B 5/443; A61B 5/4848; A61B 5/0022; A45D 44/00; A61B 2560/0242; A45D 2044/007

(57) ABSTRACT

An adaptability skincare information prompting system and an adaptability skincare prompting method are disclosed. The adaptability skincare information prompting system includes a detecting device for detecting environmental parameters of an environment where a user is currently in and physiological parameters of a target skin of the user, and an electronic device for giving skincare advice information according to the environmental parameters and then verifying or correcting the skincare advice information according to the physiological parameters.

### CROSS REFERENCE TO RELATED APPLICATIONS

This non-provisional application is a National Stage application of the International Application No. PCT/CN2014/000501, filed on May 19, 2014, which claims the priority of a China patent application No. 201410197920.7, filed on May 13, 2014.

### BACKGROUND OF THE INVENTION

Field of Invention

The invention relates to an adaptability skincare prompting system and an adaptability skincare prompting method thereof and, in particular, to an adaptability skincare prompting system and an adaptability skincare prompting method thereof that integrate a detecting device and an electronic device.

Related Art

As the progress of technology, many novel cosmetic or skincare methods have been proposed. In general, an improved skincare method includes a real-time detection. That is, the proper cosmetic and skincare approaches can be promptly provided according to the user environment or the user's state. For example, before an activity in a sun exposure environment, people tend to only select one of the commercial sunscreens with regardless the UV index (UVI), the proper sun protection factor of the selected sunscreen, or the skin status of the users. In a real-time detection system or method, the skin detection will be conducted and the detected parameters can be provided as an advice for selecting a proper skincare or beauty care product. This detection procedure can provide an advice for properly and quantitatively applying the skincare or beauty care product, which is much better than the conventional way to select and apply the skincare or beauty care product by feeling. Accordingly, this new approach can avoid the waste of the skincare or beauty care product, the burden of body, and unsatisfied cosmetic effect caused by applying insufficient skincare or beauty care product.

As mentioned above, there are many approaches for announcing the environmental UV. For example, Taiwan Patent No. 200600759 disclosed to sense the environmental UV by a UV sensing component, and then to output a recommendation for selecting a sunscreen product by a microprocessor. However, Taiwan Patent No. 200600759 does not disclose how to achieve the disclosed technology. This Taiwan Patent also disclosed to input the parameters related to the skin status and cosmetic products for adjusting the given advice of the sunscreen product. Taiwan Patent No. M355518 disclosed to integrate a UV detecting device in a mobile phone, so that the mobile phone can automatically prompt a proper protection strategy according to the detected UVI. Taiwan Patent No. M444510 disclosed a UV detecting device which stores many products with different SPF (Sun Protection Factor). The UV detecting device detects the environmental UVI and then automatically prompt one of the stored products. Taiwan Patent No. M4650640 then disclosed a wireless weather sensing system for sensing the environmental UV. This system can sense the environmental UVI and then transmit the sensed UVI to the user in a wireless manner, so that the user can have a proper sun protection based on the received data.

Besides, regarding to the skin-related physiological parameters, China Patent No. 101686819 disclosed a system and a method for real-time detecting the skin-related physiological parameters. The system and method utilize an optical based detecting device, and the optical based detecting device is communicated with an electronic device for providing physiological parameters related to the skin status and advices about using the skincare products to the users. In addition, this system and method can also upload the physiological parameters to a cloud, and the uploaded physiological parameters are then analyzed in the cloud server. Afterwards, the analyzing results can be fed back to the users as a reference or provided to a third party such as a beauty care product company or a research institute.

However, the conventional real-time detection technologies never disclose a combination of the environmental parameters and the physiological parameters. Although the concept of this combination is merely mentioned, the specific solution thereof is still not clear. In fact, the combination of the environmental parameters and the physiological parameters have very outstanding effects in this field. For example, different skin statuses can have different sunlight resistances (even in the environments of the same UVI). An example is that the skin containing high ratio of melanin can have better sun protection effect without using a high SPF product.

Moreover, the physiological parameters can be used to build a feedback mechanism for verifying the effects of the skincare or beauty care products. Taiwan patent No. 1335985 discloses to evaluating the effects of the sunscreen products based on the sunburn status after applying the sunscreen for a while. However, the feedback mechanism technology is mostly a concept description, and the specific solution is not discussed.

In addition, some technologies had the functions of detecting the environmental parameters or the physiological parameters. However, these technologies need a huge sized apparatus, which is not easily carried, for implementation. Thus, the goal of performing skincare and beauty care anyplace and anytime cannot be carried out.

### SUMMARY OF THE INVENTION

The present invention discloses an adaptability skincare prompting system and an adaptability skincare prompting method, which can provide a skincare advice to the user based on both of the environmental and physiological parameters.

Accordingly, the present invention discloses an adaptability skincare prompting system including a detecting device for detecting environmental parameters of an environment where a user is currently in and physiological parameters of a target skin of the user, and an electronic device for giving skincare advice information according to the environmental parameters and then verifying or correcting the skincare advice information according to the physiological parameters.

Preferably, the adaptability skincare prompting system further includes a cloud server communicating with the electronic device for collecting and storing the user information containing the environmental parameters and the physiological parameters from the electronic device.

Preferably, the cloud server includes a first database and a second database. The first database collects and stores the private information in the user information according to an identical code, and the second database collects and stores the non-private information in the user information according to the identical code.

Preferably, the cloud server analyzes history data of the user as a base of the skincare advice information given by the electronic device.

Preferably, the cloud server analyzes history data of another user as a base of the skincare advice information given by the electronic device.

Preferably, the adaptability skincare prompting system further includes a third-party information system communicating with the cloud server. The cloud server is able to provide the non-private information from the second database to the third-party information system.

Preferably, the skincare advice information includes product information or service information.

Preferably, the third-party information system provides the non-private information to the electronic device, and the electronic device verifies or corrects the skincare advice information accordingly.

Preferably, the detecting device has an audio connector for connecting to an audio jack of the electronic device so as to get power for driving the detecting device.

Preferably, the detecting device transmits an audio modulation signal to the electronic device for data transmission.

Preferably, the detecting device further includes a wireless transmission module, and the detecting device transmits data to the electronic device through the wireless transmission module.

In addition, the present invention also discloses an adaptability skincare prompting method, which includes the following steps of: detecting environmental parameters of an environment where a user is currently in and physiological parameters of a target skin of the user by a detecting device, and giving skincare advice information according to the environmental parameters by an electronic device, and verifying or correcting the skincare advice information according to the physiological parameters.

Preferably, the adaptability skincare prompting method further includes a step of detecting the physiological parameters to verify the effectiveness of the skincare advice information after the user implements the skincare advice information.

Preferably, the adaptability skincare prompting method further includes a step of communicating the electronic device to a cloud server and transferring the user information containing the environmental parameters and the physiological parameters from the electronic device to the cloud server. Preferably, the cloud server includes a first database and a second database. The first database collects and stores the private information in the user information according to an identical code, and the second database collects and stores the non-private information in the user information according to the identical code.

Preferably, the adaptability skincare prompting method further includes a step of analyzing history data of the user by the cloud server, which is used as a base of the skincare advice information given by the electronic device.

Preferably, the adaptability skincare prompting method further includes a step of analyzing history data of another user by the cloud server, which is used as a base of the skincare advice information given by the electronic device.

Preferably, the adaptability skincare prompting method further includes a step of communicating the cloud server to a third-party information system, and providing the non-private information from the second database to the third-party information system.

Preferably, the adaptability skincare prompting method further includes a step of providing the non-private information to the electronic device, so that the electronic device verifies or corrects the skincare advice information accordingly.

Preferably, the skincare advice information includes product information or service information.

Preferably, the adaptability skincare prompting method further includes a step of connecting an audio connector of the detecting device to an audio jack of the electronic device.

Preferably, the adaptability skincare prompting method further includes a step of transmitting an audio modulation signal from the detecting device to the electronic device for data transmission.

Preferably, the adaptability skincare prompting method further includes a step of transmitting data from the detecting device to the electronic device by wireless transmission.

Compared with the conventional technologies, the adaptability skincare prompting system and method of the invention have one or more of the following advantages:

(1) the adaptability skincare prompting system and method can provide an optimum skincare information to the user based on the results of detecting and processing the environmental and physiological parameters;

(2) the adaptability skincare prompting system and method can detect the physiological parameters, after the user implements the skincare advice information, to verify the effectiveness of the skincare advice information;

(3) the adaptability skincare prompting system and method can store the private and non-private data in the first and second databases of the cloud server, respectively, thereby increasing the security of the private data of the user;

(4) the adaptability skincare prompting system and method can further establish a communication with a third-party information system, so that it is possible to feed the status of the user back the third-party information system for future product development or correcting the advice information given by the electronic device; and

(5) the adaptability skincare prompting system and method can connect the electronic device and the detecting device by the audio connector, so that it is possible to execute the desired skincare detection and beauty care procedure anytime and anyplace.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is a block diagram showing an adaptability skincare prompting system according to a first embodiment of the invention;

FIG. 2 is a flow chart of an adaptability skincare prompting method according to the first embodiment of the invention;

FIG. 3 is a block diagram showing an adaptability skincare prompting system according to a second embodiment of the invention;

FIG. 4 is a flow chart of an adaptability skincare prompting method according to the second embodiment of the invention;

FIG. 5 is a block diagram showing an adaptability skincare prompting system according to a third embodiment of the invention;

FIG. 6 is a block diagram showing an adaptability skincare prompting system according to a fourth embodiment of the invention;

FIG. 7 is a flow chart of an adaptability skincare prompting method according to the fourth embodiment of the invention;

FIG. 8 is a schematic diagram showing the structure of a detecting device according to a fifth embodiment of the invention;

FIG. 9 is a schematic diagram showing the combination of the detecting device and an electronic device according to the fifth embodiment of the invention;

FIG. 10 is a circuit diagram of the detecting device according to the fifth embodiment of the invention;

FIG. 11 is a schematic diagram showing the original signal and the audio modulation signal outputted from the detecting device according to the fifth embodiment of the invention; and

FIG. 12 is a circuit diagram of a detecting device according to a sixth embodiment of the invention.

### DETAILED DESCRIPTION OF THE INVENTION

The present invention will be apparent from the following detailed description, which proceeds with reference to the accompanying drawings, wherein the same references relate to the same elements.

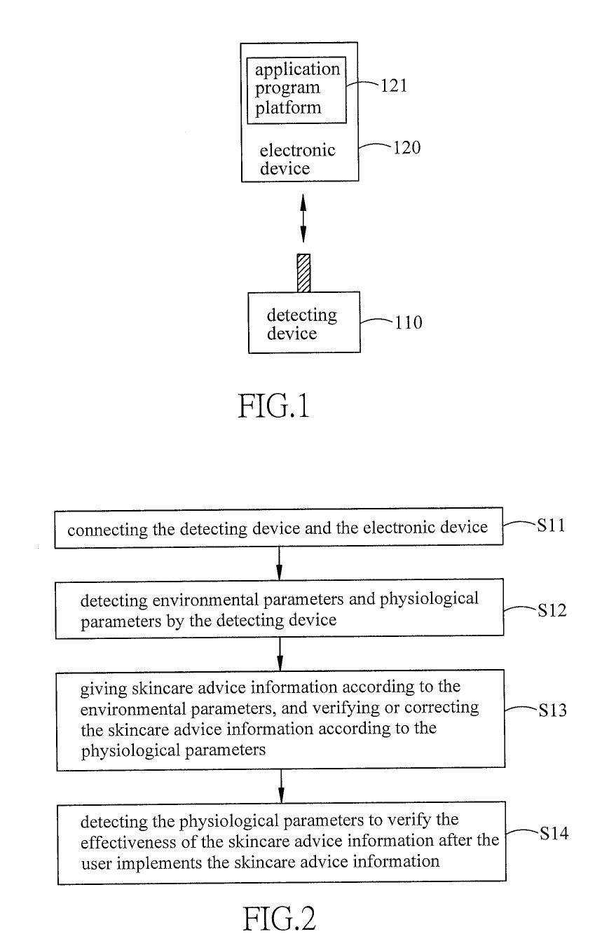

FIG. 1 is a block diagram showing an adaptability skincare prompting system according to a first embodiment of the invention. As shown in FIG. 1, the adaptability skincare prompting system includes a detecting device 110 for detecting environmental parameters of an environment where a user is currently in and physiological parameters of a target skin of the user, and an electronic device 120 for giving skincare advice information according to the environmental parameters and then verifying or correcting the skincare advice information according to the physiological parameters. The electronic device 120 can further install an application program platform 121 corresponding to the detecting device 110.

In more specific, the detecting device 110 can detect the environmental parameters such as UVI, temperature, humidity, and etc.

In practice, the detecting device 110 may include a UV detecting module, which contains a UV photodiode corresponding to the UV-B wavelength (280 nm-320 nm). To be noted, the UV-A wavelength (320 nm-400 nm) contains lower energy and has lower effect to human body, and the UVC wavelength (180 nm-280 nm) is mostly absorbed by ozone. Of course, the detectability of the UV detecting module is not limited to the above example. After receiving the UV light, the UV photodiode outputs a current signal to the microprocessor based on the intensity of the received UV light, and the microprocessor determines the UVI of the environment according to the current signal. Besides, the UV detecting module can further include a filter for blocking the visible light. Since the UV photodiode may also absorb the visible light and generate the current signal accordingly, the configuration of the filter can eliminate the effect of the visible light and thus make the detection of UVI more accurate. Moreover, the UV detecting module further includes a current buffer, a transimpedance amplifier (TIA) and an analog-to digital converter (ADC) for transforming the current signal into an analog voltage signal and then converting the analog voltage signal into a digital voltage signal, thereby further analyzing the intensity of the detected UV light. The accuracy of the UV detecting module can reach the UVI less than 1, so it is suitable for detecting not only the outdoor sunlight but also the indoor lamp light. Besides, it can be applied to a case that needs to precisely calculate the accumulated UV dose.

Moreover, the detecting device 110 may further include a temperature/humidity detecting module, which contains a temperature/humidity detecting chip. For example, the temperature/humidity detecting chip can be selected from the commercial temperature/humidity detecting chips such as the chip model number SI7013/20/21 or SHT10, 11, 15, 21. These commercial temperature/humidity detecting chips have compact size and are easily operated for detecting temperature and humidity. The temperature/humidity detecting module further includes a preset calibrating circuit for calibrating the signal outputted from the temperature/humidity detecting chip, and the calibrated signal is then transmitted to the microprocessor. This configuration allows the obtained temperature and humidity more accurate.

In specific, the detecting device 110 can detect the physiological parameters related to the skin such as the skin chromophore content, the inflammation level of the skin, the oil-water analysis of the surface skin, the skin surface imaging, and the likes. The details of the detecting device 110 will be described herein below.

Regarding to the skin chromophore content, the detecting device 110 may include a physiological optical detecting module, which contains at least one physiological photodiode and a light emitting diode (LED) for emitting the light of different wavelengths corresponding to different chromophore. The wavelength that the physiological photodiode can absorb for photoelectrical conversion is corresponding to the wavelength of the light emitted by the LED. The above-mentioned skin chromophore includes melanin, hemoglobin, collagen, fat, etc., and the chromophores have different light absorption coefficients. The LED is configured to emit light to the target skin, and the target skin reflects the light to the physiological photodiode. Since the positions of the LED and physiological photodiode are defined, the traveling length of the light passing through the skin can be determined. The absorption coefficient parameter of the target chromophore can be obtained by comparing the intensity of the light emitted from the LED and the intensity of the light absorbed by the photodiode. Then, the ratio of the target chromophore in the target skin can be calculated according to the absorption coefficient parameter. To be noted, the above-mentioned collagen has good directivity, so it is necessary to apply two lights from different directions for calculating the correct absorption coefficient of the collagen chromophore. The needed two lights from different directions can be generated by configuring two LED (a first LED and a second LED) and one physiological photodiode. For example, the first LED, the physiological photodiode and the second LED are arranged with included angles of 90 degrees, and the traveling lengths of the lights emitted from the first and second LEDs to the target skin are substantially equal to the traveling lengths of the lights reflected from the target skin to the physiological photodiode.

The theory of detecting the inflammation is similar to the detection of skin chromophore content. Since the inflammation of the target skin will lead to the proliferation or expansion of microvascular, and the absorption coefficient of the hemoglobin in the chromophore and the density of microvascular have positive correlation, it is possible to determine the inflammation of the target skin according to the absorption coefficient of hemoglobin. Different from the above detection of skin chromophore content, the inflammation is determined by comparing the absorption coefficients of hemoglobin of the target skin and the skin around the target skin. Accordingly, the detecting device 110 must detect at least two positions of the skin for determining the inflammation of the target skin. The detection method can be referred to the above configuration of the LED and physiological photodiode of the physiological optical detecting module. For example, it is possible to configure multiple LEDs and physiological photodiodes, which correspond to the absorption wavelength of hemoglobin and are arranged in an array, for obtaining the absorption coefficients of hemoglobin corresponding to the large-sized target skin. Then, the inflammation condition of the target skin can be determined by comparing the absorption coefficients of hemoglobin of the target skin and the skin around the target skin.

For the oil-water analysis of the surface skin, the detecting device 110 includes a physiological resistance module, which includes a pair of electrodes (a first electrode and a second electrode). The first electrode and the second electrode have a preset voltage gap. When the pair of electrodes contact the target skin, a micro current flows from the first electrode to the second electrode through the target skin. Since the resistances of oil and water in the surface skin are different, the distribution of oil and water in the target skin can be detected by analyzing the micro current.

For the skin surface imaging, the detecting device 110 includes a skin imaging module, which has a lens with high resolution for shooting the image of the target skin. After analyzing the image of the target skin, the skin information, such as the pore size, the quality of skin texture, the pigmentation of the surface skin, and the likes, can be obtained.

FIG. 2 is a flow chart of an adaptability skincare prompting method according to the first embodiment of the invention, and the adaptability skincare prompting method is applied to the adaptability skincare prompting system of FIG. 1. The adaptability skincare prompting method includes the steps of: connecting the detecting device 110 and the electronic device 120 (S11); detecting environmental parameters of an environment where a user is currently in and physiological parameters of a target skin of the user by the detecting device 110 (S12); and giving skincare advice information according to the environmental parameters by the electronic device 120, and verifying or correcting the skincare advice information according to the physiological parameters by the electronic device 120 (S13). The step S11 of connecting the detecting device 110 and the electronic device 120 is to establish a communication between the detecting device 110 and the electronic device 120.

The following description is an example that uses the adaptability skincare prompting system and method in the sun protection. Firstly, the step S11 is to connect the detecting device 110 and the electronic device 120. Next, the UV detecting module of the detecting device 110 retrieves the environmental UVI, and the physiological optical detecting module of the detecting device 110 retrieves the chromophore concentrations of melanin and collagen of the user (step S12). After receiving the environmental parameters and the physiological parameters, the electronic device 120 recommends a proper sunscreen product based on the environmental parameters, and then provides an effective period of the sunscreen product according to the physiological parameters (step S13). For instance, the electronic device 120 is installed an application program platform 121 corresponding to the detecting device 110. After establishing the communication of the detecting device 110 and the electronic device 120, the UV detecting module of the detecting device 110 retrieves the environmental UVI by 5, and the physiological optical detecting module of the detecting device 110 retrieves the chromophore concentrations of melanin of the user by 1.6% and the chromophore concentrations of collagen of the user by 20%. In this case, the electronic device 120 recommends a sunscreen product of SPF 30 based on the detected UVI. The detected chromophore concentrations of melanin and collagen are used to compare with the database of the application program platform 121 or calculated with a proper function so as to obtain the longest time that the user is exposed under the environment (UVI=1) and still has no sunburn. That is, the time to accumulate the UV dose to the minimal erythermal dose (MED) under the standard environment (UVI=1). In the case that the chromophore concentrations of melanin of the user is 1.6%, the chromophore concentrations of collagen of the user is 20%, and the electronic device 120 calculates and obtains that the duration time of the user's skin in the UV environment is 100 minutes, the electronic device 120 further calculates as follow: 100/5*30=600. This calculation result is added to the skincare advice information to indicate the user that effective time of the sunscreen product (SPF 30) is 600 minutes (or 10 hours). Of course, the above example is not to limit the invention. In other cases, the user can input a favored sunscreen product through the application program platform 121, and then the electronic device 120 can provide an effective time according to the function stored in the application program platform 121.

The following description is an example that uses the adaptability skincare prompting system and method in the moisturizing application. Firstly, the detecting device 110 is connected with the electronic device 120 (step S11). Next, the temperature/humidity detecting module of the detecting device 110 retrieves the environmental temperature and humidity, and the physiological optical detecting module of the detecting device 110 retrieves the chromophore concentrations of collagen of the user (step S12). After receiving the environmental parameters and the physiological parameters, the electronic device 120 recommends a proper moisturizing product based on the environmental parameters, and then provides a modified using recommendation of the moisturizing product according to the physiological parameters (step S13).

To be noted, the adaptability skincare prompting system and method of the invention can also provide the recommendations based on other parameters retrieved by other functions of the electronic device 120. For example, in the sun protection application, the electronic device 120 may have a GPS function and a network function, which can combine to obtain the altitude parameter of the user. Then, the electronic device 120 uses the altitude parameter to verify or correct the skincare advice information. In more specific, if the user is positioned at the altitude of 3000 meters, the intensity of UV light increases about 50%. Thus, the UVI measured by the UV detecting module must be multiplied by 1.5, and the electronic device 120 corrects the skincare advice information based on the modified UVI. For example, the electronic device 120 can recommend a sunscreen product with higher SPF.

As shown in FIG. 2, the adaptability skincare prompting method further includes a step of detecting the physiological parameters to verify the effectiveness of the skincare advice information after the user implements the skincare advice information (step S14).

In practice, the detection of physiological parameters can not only correct the skincare advice information in advance but also verify the effect of performing the skincare advice information later. Taking sun protection as an example, the user can apply the proper sunscreen product based on the skincare advice information provided by the step S13, and then execute the step S14 at the next day. The step S14 is to analyze the image obtained by the skin imaging module of the detecting device 110 or to analyze the melanin chromophore concentration obtained by the physiological optical module of the detecting device 110, thereby determining the sun exposure status and evaluating the effect of the sunscreen product. Taking moisturizing as an example, the step S14 can be performed, after waiting for a preset period as the step S13 has been finished, to quantitatively evaluate the moisturizing effect of the moisturizing product according to the collagen obtained by the physiological optical module of the detecting device 110.

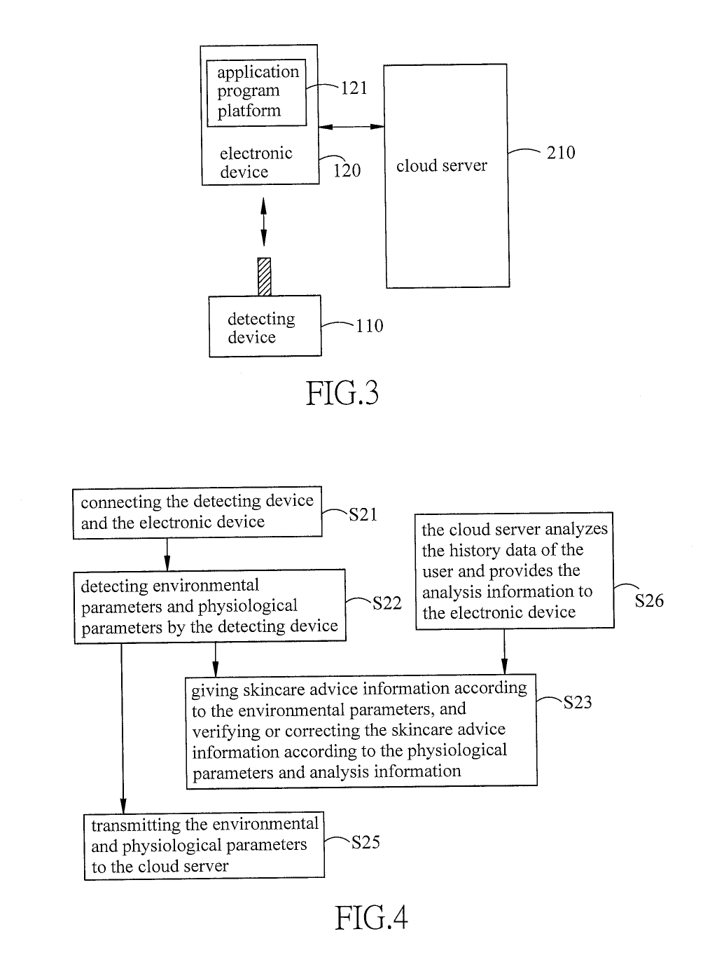

FIG. 3 is a block diagram showing an adaptability skincare prompting system according to a second embodiment of the invention. As shown in FIG. 3, the adaptability skincare prompting system further includes a cloud server 210, which communicates with the electronic device 120 for collecting and storing the user information containing the environmental parameters and the physiological parameters from the electronic device 120.

FIG. 4 is a flow chart of an adaptability skincare prompting method according to the second embodiment of the invention, and the adaptability skincare prompting method is applied to the adaptability skincare prompting system of FIG. 3. The steps S21, S22 and S23 can be referred to the steps S11, S12 and S13 of the adaptability skincare prompting method in the first embodiment. Different from the first embodiment, the adaptability skincare prompting method of FIG. 4 includes a step S24 for communicating the electronic device to the cloud server 210 and transferring the user information containing the environmental parameters and the physiological parameters to the cloud server 210. The cloud server 210 analyzes the history data of the user, and the analyzing result can be provided to the electronic device 120 (step S25). Accordingly, the electronic device 120 gives the skincare advice information based on the environmental parameters and physiological parameters in view of the history data from the cloud server 210. The steps S25 and S26 will be further described hereinafter.

In the step S25, the electronic device 120 is communicated with the cloud server 210, so that the cloud server 210 can collect the data that the user utilizes the adaptability skincare prompting system, such as the environmental parameters and physiological parameters of all detections. These data can help the system manager to develop and modify the entire system. For example, the approach that the application program platform of the electronic device 120 provides the skincare advice information can be updated, so that the user can get a better skincare advice. In addition, not all data processes are handled by the electronic device 120. That is, some complex processes can be handled and analyzed by the cloud server 210, and the results are sent back from the cloud server 210 to the electronic device 120, so that the electronic device 120 can properly provide the skincare advice information.

In the step S26, the user can operate the electronic device 120 to retrieve the history records of utilizing the adaptability skincare prompting system from the cloud server 210. This history records can be used as references of the long-term beauty care strategy. The above steps can be achieved by a normal smart phone (electronic device 120), and preferably the electronic device 120 is personal use only. This privacy application can be carried out by using the adaptability skincare prompting system to register or login the application program platform 121, or by comparing the physiological parameters, which were sent from the device and stored in the cloud server 210, so as to determine whether the user is the same one. When the user uses the adaptability skincare prompting system for long term, the all history records become meaningful and can be used as the reference of personal beauty care. For example, the detecting device 110 can cooperate with the application program platform 121 for tracking the melanin concentration variation of the target skin, thereby verifying whether the applied skincare or beauty care products are useful. Another example is to track the inflammation of the target skin. If the inflammation is not cured or improved and the other physiological parameter (e.g. the melanin concentration) also has irregular variation, the system can issue a warning message to announce a skin lesion potential, so that the user can contact a doctor earlier. An additional example is to combine the environmental parameters and the physiological parameters in the long-term detection. In one case, the skin imaging module detects that the skin may have erytherma due to heat and humidity environment, and the history records indicate that the environment conditions causing the erytherma are higher than a certain temperature and humidity. Accordingly, the electronic device 120 will connect to the local weather broadcasting institute to obtain the predicted temperature and humidity. If the obtained predicted temperature and humidity are higher than the certain temperature and humidity capable of causing the erytherma, the electronic device 120 issues a warning message to the user to remind the user to take care of the skin condition. Furthermore, the cloud server 210 can perform a statistical analysis according to the history records of the user and then provide the analyzing result to the user by, for example, a chart. Accordingly, the user can track and manage the variation of the history records.

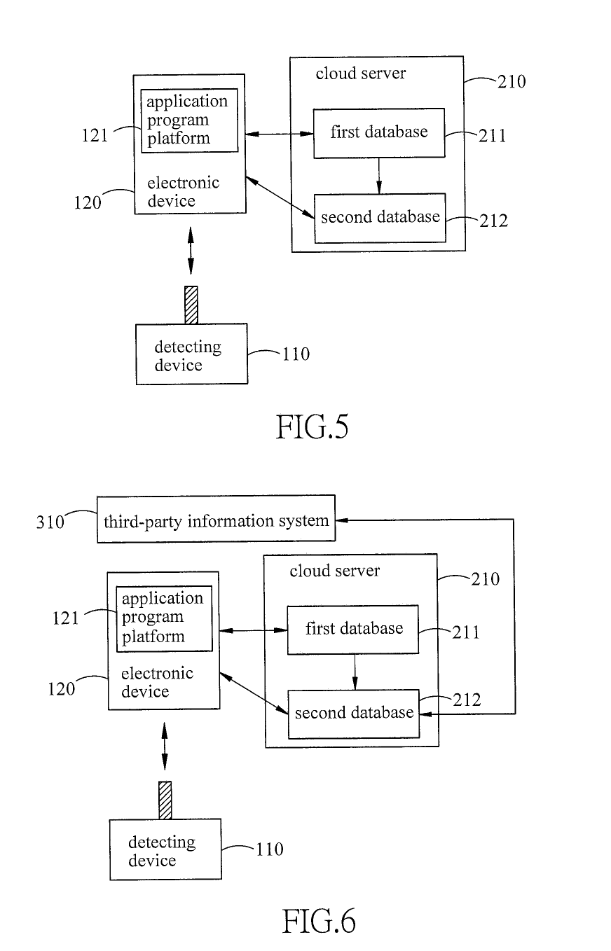

FIG. 5 is a block diagram showing an adaptability skincare prompting system according to a third embodiment of the invention. As shown in FIG. 5, the cloud server 210 includes a first database 211 and a second database 212. The first database 211 collects and stores the private information in the user information according to an identical code, and the second database 212 collects and stores the non-private information in the user information according to the identical code.

In more detailed, the electronic device 120 can include a user identical code (UID) in the user information and then transfer the user information to the cloud server 210. The UID is corresponding to the electronic device 120. When the cloud server 210 receives the user information containing the UID at the first time, it will collect and store the private part of the user information (e.g. the password for registering the application program platform) in the first database 211. Then, the non-private part of the user information (e.g. the environmental or physiological parameters) is collected and stored in the second database 212. When the cloud server 210 receives the user information containing the same UID at the second time, it compares and figures out that this UID information has been stored in the first database 211. Then, the cloud server 210 will directly store the non-private information in the second database 212. Accordingly, the private and non-private information of the user information can be separately stored, which is benefit in the future information security management.

In addition, the cloud server 210 can analyze the history data of another user and provide the analyzing result as the reference of the electronic device 120 for giving the skincare advice information.

Since the private and non-private data of the user information are stored in the first database 211 and the second database 212, respectively, the cloud server 210 can analyze the non-private data of other users stored in the second database 212 without invasion others privacy. For example, when the user starts to use a new beauty care product, the cloud server 210 analyzes the environmental or physiological parameters stored in the second database 212, which correspond to the user using the same product, and then provides the analyzing result to the user. Accordingly, the user can estimate the effective time this beauty care product. In another example, since the durability to UV light of human body may increase as the accumulation of exposure time under the sun, the cloud server 210 can analyze the environmental or physiological parameters stored in the second database 212, which correspond to the similar users having tanning plans, and then provides the analyzing result, which contains the minimal erythermal dose and melanin concentration variations, to the user. Thus, the user can make his/her personal tanning plan accordingly. The above step is included in the step S26, so the details are not shown in the figures.

FIG. 6 is a block diagram showing an adaptability skincare prompting system according to a fourth embodiment of the invention. As shown in FIG. 6, the adaptability skincare prompting system further includes a third-party information system 310 communicating with the cloud server 210. The cloud server 210 is able to provide the non-private information from the second database 212 to the third-party information system 310.

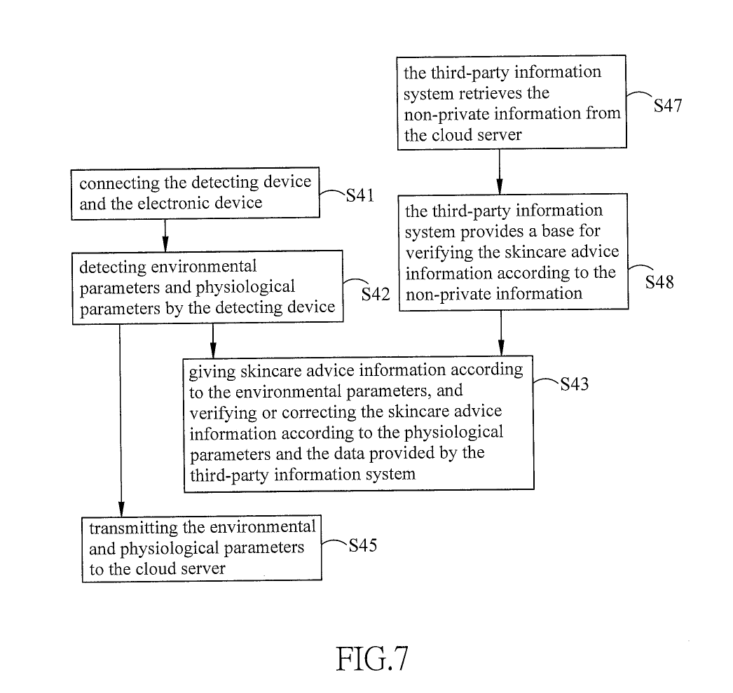

FIG. 7 is a flow chart of an adaptability skincare prompting method according to the fourth embodiment of the invention, and the adaptability skincare prompting method is applied to the adaptability skincare prompting system of FIG. 6. The steps S41, S42, S43 and S45 can be referred to the steps S21, S22, S23 and S25 of the adaptability skincare prompting method in the second embodiment. Different from the second embodiment, the adaptability skincare prompting method of the third embodiment includes steps of: the cloud server 210 retrieves the non-private information from the second database 212 and provides the non-private information to the third-party information system 310 (step S47); and the third-party information system 310 provides the non-private information to the electronic device 120, so that the electronic device 120 verifies or corrects the skincare advice information accordingly (step S48). Moreover, in the step S43, the electronic device 120 can give a modified skincare advice information according to the environmental parameters, the physiological parameters and the correction reference provided from the third-party information system in the step S48. The steps S47 and S48 will be described in details hereinafter.

In the step S47, since the third-party information system 310 can only access the second database 212 to retrieve the non-private data, the personal private data of the users can be secured so as to prevent the personal private data leakage. The third-party information system 310 can be the manufacturers, distributers or researching institutes of the beauty care products, and the data retrieved from the second database 212 can be used in new products development, market research or theory correction. The cloud server 210 can execute the statistical analysis of the data of the second database 212 and then transmit the analyzing results to the third-party information system 310. Alternatively, the third-party information system 310 may input a search keyword to search the interesting data in the second database 212. To be noted, the method for providing the data from the second database 212 of the cloud server 210 to the third-party information system 310 is not limited to the above examples.

In the step S48, the third-party information system 310 receives and analyzes the data from the second database 212 and then transmits the feedback information to the electronic device 120, so that the electronic device 120 can provide proper skincare advice information to the user. In practice, the products with the same effect labels but manufactured by different manufacturers (e.g. the sunscreen products with the same SPF) may provide different skincare or beauty care results. In this case, the electronic device 120 provides the skincare advice information according to the conditions or parameters provided by the third-party information system 310 of the manufacturers or research institutes of beauty care products. Accordingly, the third-party information system 310 can modify the conditions or parameters according to the retrieved data, thereby making the entire system more flexible and close to the reality. For example, the third-party information system 310 is a beauty care product manufacturer, and the user is using the moisturizing products of the manufacturer. The third-party information system 310 analyzes the data from the second database 212, which refer to the similar conditions and the products, and then makes a conclusion that this moisturizing product has shorter effective period in the similar environment. Accordingly, the third-party information system 310 issues a correct message to the electronic device 120 of the user, and the corrected message (corrected skincare advice information) will suggest the user to increase the frequency of using the moisturizing product for achieving the desired moisturizing effect. As a result, the skincare advice information is provided with referring to the environmental parameters, the physiological parameters and the message from the third-party information system 310 (see step S43 of FIG. 7).

Besides, the skincare advice information may include the product information or service information.

In this case, when the adaptability skincare prompting system is cooperated with a specific company, the third-party information system 310 is an information window of the company with respect to the adaptability skincare prompting system. The skincare advice information provided by the electronic device 120 includes one or more suggested products of this company according to the detected environmental or physiological parameters. In more detailed, the electronic device 120 can search the similar data from the second database 212 according to the detected environmental or physiological parameters, and then provide the skincare advice information including the suggested products of this company that other users have used at the similar conditions. To be noted, the method for suggesting the products by the skincare advice information is not limited. For example, the specific beauty care products can be registered in the application program platform 121 of the electronic device 120, and the contents of the registered products are determined. The electronic device 120 will prompt the proper beauty care products as well as the suggested dose of the product every time to the user. If the user applies the product according to the suggested dose, the application program platform can record the remained amount of the beauty care product. Then, it can announce the user to buy a new one as the record indicates that the product is going to run out, or suggest the user to change a larger/smaller sized product. The skincare advice information includes not only the product information but also the related beauty care services. For example, the specific company of the third-party information system 310 may also run some medical beauty care services. When the detecting device 110 detects the physiological parameters of the user, the electronic device 120 can add the medical beauty care services of the specific company in the skincare advice information after analyzing the physiological parameters.

Furthermore, the user information transmitted from the electronic device 120 to the cloud server 210 may contain the feedbacks of the user related to the product or service information of the skincare advice information. For example, the product or service information can be shown on the screen of the electronic device 120 as a hyperlink as the electronic device 120 provides the skincare advice information to the user. Besides, the user information transmitted from the electronic device 120 to the cloud server includes the click amount of the hyperlink, which is determined as a non-private data and stored in the second database 212. The third-party information system 310 is communicated with the cloud server 210 for retrieving the click amount data, which are used as the reference of future business strategy or market investigation.

If the third-party information system 310 corresponds to a specific company, the application program platform 121 of the electronic device 120 can give virtual points to the user based on the operation of the detecting device 110 or the registration of the beauty care products of the company. This method can rise the interesting of the user to the adaptability skincare prompting system, thereby increasing the dependency and reliability of the user to the adaptability skincare prompting system. The user can spend the virtual points to exchange the products or services provided by the company, or to get the discounts of the products or services provided by the company. In addition, the application program platform 121 of the electronic device 120 can be built in the electronic store of the company, so that the user can easily purchase the products of the specific company or spend the virtual points.

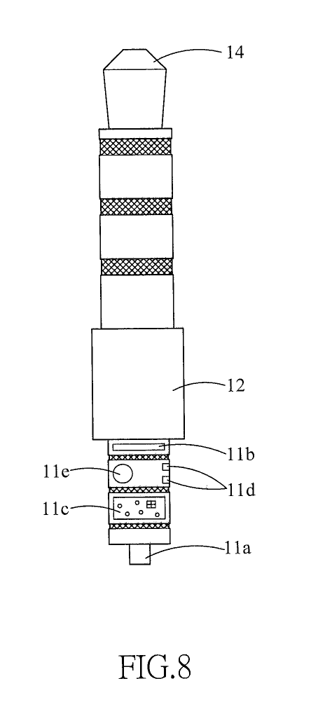

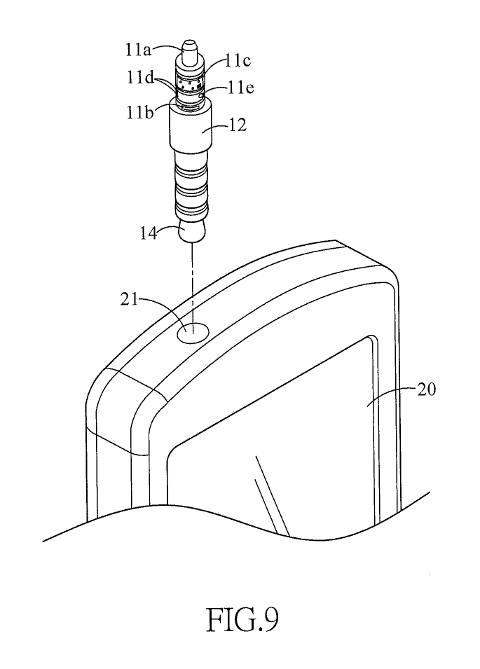

FIG. 8 is a schematic diagram showing the structure of a detecting device according to a fifth embodiment of the invention. As shown in FIG. 8, the detecting device 10 includes a UV detecting module 11a, a temperature/humidity detecting module 11b, a physiological optical module 11c, a physiological resistance module 11d, a skin imaging module 11e, a microprocessor 12 and an audio connector 14. FIG. 9 is a schematic diagram showing the combination of the detecting device and an electronic device according to the fifth embodiment of the invention. As shown in FIG. 9, the detecting device 10 uses the audio connector 14 to connect to an audio jack 21 of the electronic device 20 so as to get power for driving the detecting device 10. In this case, each of the steps S11, S21 and S21 is to connect the audio connector 14 of the detecting device 10 to the audio jack 21 of the electronic device 20 so as to communicate the detecting device 10 to the electronic device 20.

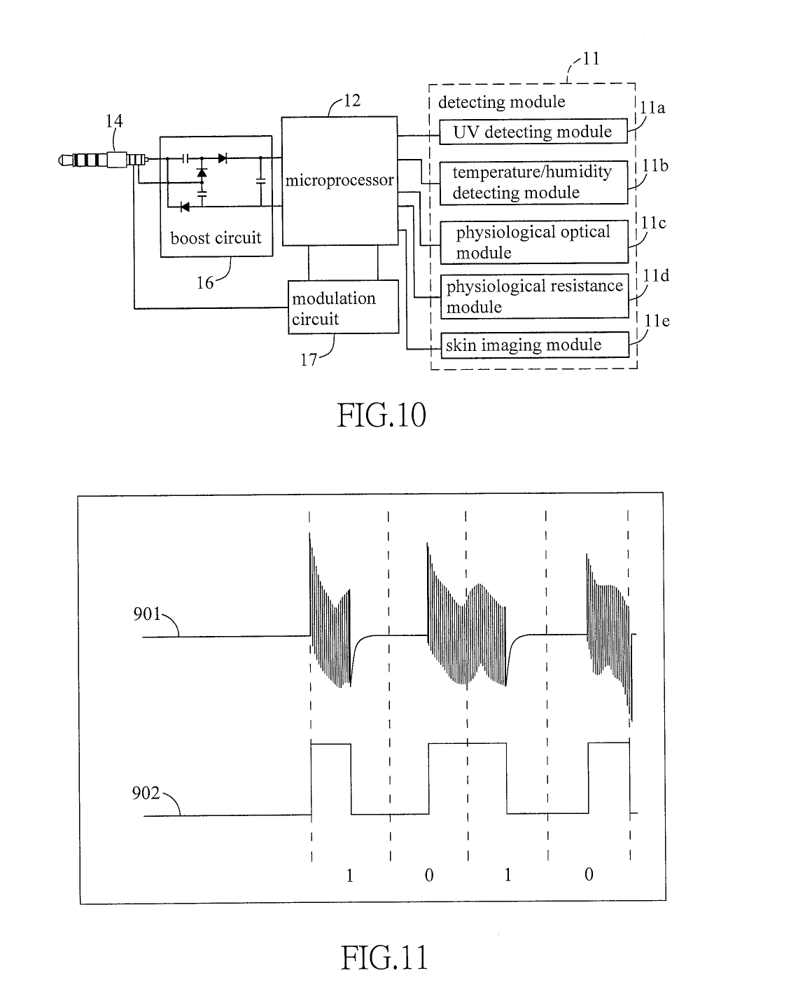

In order to make the user to use the adaptability skincare prompting system anytime and anyplace, it is necessary to minimize the size of the detecting device 10 and make the detecting device 10 portable. In this case, the electronic device 20 can be a smart phone, a tablet computer or other personal mobile electronic device. The detecting device 10 uses the audio connector 14 to connect to an audio jack 21 of the electronic device 20 so as to get power from the electronic device 20 for driving the detecting device 10. The application program platform of the electronic device 20 can lock the output signal of the audio jack 21 at the maximum value so as to provide enough voltage to the detecting device 10. Accordingly, the detecting device 10 can operate without using an independent power source, thereby achieving the desired goals of minimization and portable. In practice, even the output signal of the audio jack 21 is locked at the maximum value by the application program platform, the output voltage from the audio jack 21 of a general smart phone (the electronic device 20) is not enough to drive the microprocessor 12. As shown in FIG. 10, which is a circuit diagram of the detecting device according to the fifth embodiment of the invention, the audio connector 14 and the microprocessor 12 are connected through a boost circuit 16. The boost circuit 16 has the rectifying function for converting an AC signal from the audio connector 14 into a DC signal, which is accepted by the microprocessor 12, and increasing the voltage of the DC signal for driving the microprocessor 12. The configuration of the boost circuit 12 shown in FIG. 10 is only an illustrated aspect, and it is not to limit the present invention. In this case, the boost circuit 16 can increase the voltage of the AC signal from the audio connector 14 to triple of voltage. For example, if the signal from the audio connector 14 is a sine wave with the amplitude of 1V, the boost circuit 16 can convert it to a 3V DC signal. Although the detecting device 10 has the boost circuit 16 for boosting the voltage, the output voltage from the audio jack 21 of some electronic devices 20 is still not enough to drive the general microprocessor 12. In this case, it is suggested to select a micro processing chip containing a DC/DC convertor, thereby further increasing the voltage of the DC signal from the boost circuit 16 to be capable of driving the microprocessor 12.

To be noted, the connecting method of the detecting device 10 and the electronic device 20 is not limited to the above example. For example, the detecting device 10 and the electronic device 20 can also be connected through a USB connector or other connecting method. Alternatively, the detecting device 10 may have an independent power source, so the detecting device 10 is only to communicate with the electronic device 20 without retrieving the power source from it.

As shown in FIG. 10, the detecting module 11 containing the UV detecting module 11a, the temperature/humidity detecting module 11b, the physiological optical module 11c, a physiological resistance module 11d and the skin imaging module 11e is connected to the microprocessor 12 for providing the detected parameters to the microprocessor 12. To be noted, the detecting module 11 of FIG. 10 is for an illustration only, and the detecting module 11 of the detecting device 10 of the invention does not need to contain all of the above modules. In practice, the detecting module 11 only needs some modules for detecting the environmental parameters and some modules for detecting the physiological parameters. For example, the detecting module 11 may include the UV detecting module 11a for detecting the UVI and the physiological optical module 11c for detecting the skin chromophore concentration, or include the temperature/humidity detecting module 11b for detecting the temperature and humidity and the skin imaging module 11e for detecting the surface skin status. Of course, the detecting module 11 can also contain other detecting modules for detecting other environmental and physiological parameters.

If the detecting device 10 and the electronic device 20 are connected through the connection of the audio connector 14 and the audio jack 21, the detecting device 10 can also transmit signal to the electronic device 20 through this connection. In this case, the signal transmitted to the electronic device 20 is similar to the signal inputted by a normal microphone. Referring to FIG. 10, since the electronic device 20 may not successfully identify the signal from the audio jack 21, which is not similar to the normal audio signal, the detecting device 10 further includes a modulation circuit 17 for modulating the input signal to generate the qualified signal that can be identified by the electronic device 20. In more detailed, as shown in FIG. 11, the microprocessor 12 encodes the signal to be transmitted to the electronic device 20 by Manchester encoding. That is to define the signal from high to low within a certain period is a logic “1”, and the signal from low to high within a certain period is a logic “0”. As shown in FIG. 11, the original signal 902 can be encoded into a string of “1010”. Afterwards, the microprocessor 12 transmits the original signal 902 to the modulation circuit 17, and the modulation circuit 17 loads the high level of the original signal 902 to the signal to be identified by the audio jack 21 of the electronic device 20. Herein, the signal is an 8 k Hz signal, and the low level of the original signal 902 is zero. Accordingly, as shown in FIG. 11, the audio modulation signal 901 is obtained and transmitted to the audio jack 21 through the audio connector 14. After the electronic device 20 receives the audio modulation signal 901, the application program platform of the electronic device 20 decodes the received audio modulation signal 901 so as to obtain the desired data transmitted through the original signal 902. Of course, this invention is not limited to the above approach of transmitting the signal to the electronic device 20 through the connection of the audio connector 14 and the audio jack 21. For example, the modulation circuit 17 can load the high level of the original signal 902 based on a frequency other than the above-mentioned 8 k Hz.

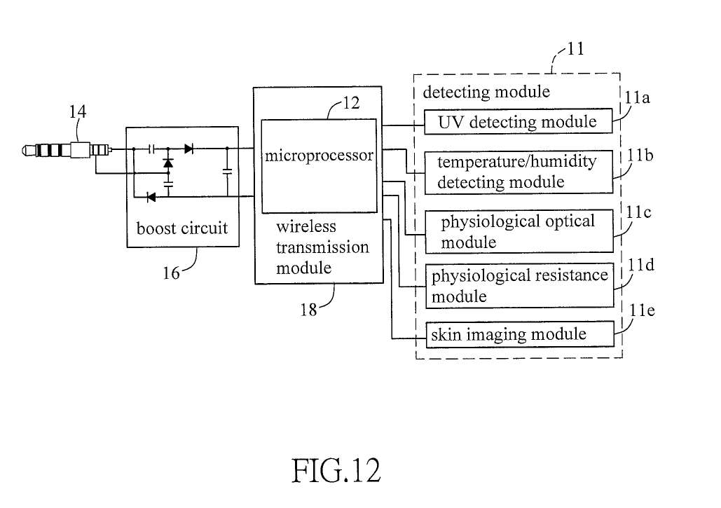

However, the above approach of using the audio jack 21 of the electronic device 20 to transmit signal is limited by the maximum identical frequency of the audio jack 21 of the electronic device 20, which is about tens kHz. This limitation is not suitable for transmitting large-sized data such as the image retrieved by the skin imaging module 11e. In order to improve the data transmission efficiency, the detecting device 10 may provide the signal to the electronic device 20 by wireless transmission. FIG. 12 is a circuit diagram of a detecting device according to a sixth embodiment of the invention. As shown in FIG. 12, the detecting device 10 includes a wireless transmission module 18 instead of the modulation circuit 17. The wireless transmission module 18 can be a Bluetooth module, a WiFi module, a near-field communication (NFC) module, or the likes. In this embodiment, the wireless transmission module 18 is a Bluetooth module, and the microprocessor 12 is a micro processing chip installed with a Bluetooth module. Since the wireless transmission (e.g. the Bluetooth transmission) can achieve the data transmission up to 1M bits per second, the detecting device 10 can transmit a large amount of data to the electronic device 20 in a short time through the wireless transmission module 18, thereby further improving the data transmission efficiency.

Moreover, the detecting device 10 may further include a protecting member. For example, the detecting device 10 includes an audio connector cover for covering the audio connector 14 of the detecting device 10 as the detecting device 10 is not connected to the electronic device 20. Thus, the audio connector 14 can be protected from the dirt or moisture in the environment, thereby keeping the transmission function of the audio connector 14. Similarly, the detecting device 10 may also include a detecting module cover for covering the exposed parts of the detecting module as the detecting module is not in use. Thus, the detecting module cover can protect the detecting from the dirt or moisture in the environment, thereby keeping the detecting function of the detecting module. The detecting device 10 may have a long shape, and the audio connector cover and the detecting module cover can be designed based on the long shape of the detecting device 10 to make the device more beautiful or easily carry out. Furthermore, when the detecting device 10 includes the UV detecting module 11a, the part of the detecting module cover corresponding to the detecting module can be manufactured with a transparent material, so that the detecting device 10 can detect the UVI without opening the cover.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. An adaptability skincare prompting system, comprising:
a detecting device for detecting environmental parameters of an environment where a user is currently in and physiological parameters of a target skin of the user; and
an electronic device for giving skincare advice information according to the environmental parameters and then verifying or correcting the skincare advice information according to the physiological parameters.

2. The adaptability skincare prompting system of claim 1, further comprising:
a cloud server communicating with the electronic device for collecting and storing user information containing the environmental parameters and the physiological parameters from the electronic device.

3. The adaptability skincare prompting system of claim 2, wherein the cloud server comprises a first database and a second database, the first database collects and stores private information in the user information according to an identical code, and the second database collects and stores non-private information in the user information according to the identical code.

4. The adaptability skincare prompting system of claim 3, wherein the cloud server analyzes history data of the user as a base of the skincare advice information given by the electronic device.

5. The adaptability skincare prompting system of claim 3, wherein the cloud server analyzes history data of another user as a base of the skincare advice information given by the electronic device.

6. The adaptability skincare prompting system of claim 3, further comprising:
a third-party information system communicating with the cloud server, wherein the cloud server provides the non-private information from the second database to the third-party information system.

7. The adaptability skincare prompting system of claim 6, wherein the skincare advice information comprises product information or service information.

8. The adaptability skincare prompting system of claim 6, wherein the third-party information system provides the non-private information to the electronic device, and the electronic device verifies or corrects the skincare advice information according to the non-private information.

9. The adaptability skincare prompting system of claim 1, wherein the detecting device has an audio connector for connecting to an audio jack of the electronic device so as to get power for driving the detecting device from the electronic device.

10. The adaptability skincare prompting system of claim 9, wherein the detecting device transmits an audio modulation signal to the electronic device for data transmission.

11. The adaptability skincare prompting system of claim 9, wherein the detecting device further comprises a wireless transmission module, and the detecting device transmits data to the electronic device through the wireless transmission module.

12. An adaptability skincare prompting method, comprising following steps of:
detecting environmental parameters of an environment where a user is currently in and physiological parameters of a target skin of the user by a detecting device;
giving skincare advice information according to the environmental parameters by an electronic device; and
verifying or correcting the skincare advice information according to the physiological parameters.

13. The adaptability skincare prompting method of claim 12, further comprising a step of:
detecting the physiological parameters to verify an effectiveness of the skincare advice information after the user implements the skincare advice information.

14. The adaptability skincare prompting method of claim 13, further comprising a step of:
communicating the electronic device to a cloud server and transferring user information containing the environmental parameters and the physiological parameters from the electronic device to the cloud server.

15. The adaptability skincare prompting method of claim 14, wherein the cloud server comprises a first database and a second database, the first database collects and stores private information in the user information according to an identical code, and the second database collects and stores non-private information in the user information according to the identical code.

16. The adaptability skincare prompting method of claim 15, further comprising a step of:
analyzing history data of the user by the cloud server, which is used as a base of the skincare advice information given by the electronic device.

17. The adaptability skincare prompting method of claim 15, further comprising a step of:
analyzing history data of another user by the cloud server, which is used as a base of the skincare advice information given by the electronic device.

18. The adaptability skincare prompting method of claim 15, further comprising a step of:
communicating the cloud server to a third-party information system, and providing the non-private information from the second database to the third-party information system.

19. The adaptability skincare prompting method of claim 18, further comprising a step of:
providing the non-private information to the electronic device, so that the electronic device verifies or corrects the skincare advice information according to the non-private information.

20. The adaptability skincare prompting method of claim 18, wherein the skincare advice information comprises product information or service information.

21. The adaptability skincare prompting method of claim 12, further comprising a step of:
connecting an audio connector of the detecting device to an audio jack of the electronic device.

22. The adaptability skincare prompting method of claim 21, further comprising a step of:
transmitting an audio modulation signal from the detecting device to the electronic device for data transmission.

23. The adaptability skincare prompting method of claim 21, further comprising a step of:
transmitting data from the detecting device to the electronic device by wireless transmission.
